SEQUENCE LISTING (1 sequence)

SEQ ID NO: 1 - protein, 435 aa, Propionibacterium acnes
MSISKDSRIAIIGAGPAGLAAGMYLEQAGFHDYTILERTDHVGGKCHSPNYHGRRYEMGAIMGVPSYDTIQEIMDRTGDKVDGPKLRREFLHEDGEIYVPEKDPVRGPQVMAAVQKLGQLLATKYQGYDANGHYNKVHEDLMLPFDEFLALNGCEAARDLWINPFTAFGYGHFDNVPAAYVLKYLDFVTMMSFAKGDLWTWADGTQAMFEHLNATLEHPAERNVDITRITREDGKVHIHTTDWDRESDVLVLTVPLEKFLDYSDADDDEREYFSKIIHQQYMVDACLVKEYPTISGYVPDNMRPERLGHVMVYYHRWADDPHQIITTYLLRNHPDYADKTQEECRQMVLDDMETFGHPVEKIIEEQTWYYFPHVSSEDYKAGWYEKVEGMQGRRNTFYAGEIMSFGNFDEVCHYSKDLVTRFFVAAALEHHHHHH

(19) United States
(12) Patent Application Publication
(10) Pub. No.: US 2024/0409967 A1
(43) Pub. Date: Dec. 12, 2024

(54) CHEMOENZYMATIC SYNTHESIS OF SEBACIC ACID AND DIBUTYL SEBACATE

(71) Applicant: The Government of the United States of America, as represented by the Secretary of the Navy, Arlington, VA (US)

(72) Inventors: Gregory A. Ellis, Rockville, MD (US); Igor L. Medintz, Springfield, VA (US); Alfred G. Stern, Upper Marlboro, MD (US); Adam A. Meares, Springfield, VA (US); Aaron D. Smith, Alexandria, VA (US)

(73) Assignee: The Government of the United States of America, as represented by the Secretary of the Navy, Arlington, VA (US)

(21) Appl. No.: 18/737,735

(22) Filed: Jun. 7, 2024

Related U.S. Application Data
(60) Provisional application No. 63/507,556, filed on Jun. 12, 2023.

(51) Int. Cl. C12P 7/44; C12N 9/20; C12N 9/90

(52) U.S. Cl. CPC C12P 7/44; C12N 9/20; C12N 9/90; C12Y 301/01003; C12Y 502/01005

(57) ABSTRACT

Described herein is a chemoenzymatic route to produce the small molecule sebacic acid from the renewable feedstock, e.g. vegetable oil. In one embodiment, this involves treating the a starting material such vegetable oil with KMnO₄, sodium thiocyanate, and acid to obtain sebacic acid. Yield could be improved by pretreating the starting material with a lipase and then with a linoleic acid isomerase obtained from Propionibacterium acnes to obtain trans-10, cis-12 conjugated linoleic acid (CLA). The sebacic acid can be further processed with lipase and 1-butanol to obtain dibutyl sebacate.

### REFERENCE TO RELATED APPLICATIONS

This application claims the benefit of U.S. Provisional Application No. 63/507,556, filed on Jun. 12, 2023. The provisional application and all other publications and patent documents referred to throughout this nonprovisional application are incorporated herein by reference. This application incorporates by reference the Sequence Listing XML file submitted herewith via the patent office electronic filing system having the file name “211314-US2.xml” with a file size of 2, 166 bytes.

### TECHNICAL FIELD

The present disclosure is generally related to synthesis of sebacic acid.

### DESCRIPTION OF THE RELATED ART

The small molecule sebacic acid, also known as 1,10-decanedioic acid, CAS Registry number 111-20-6, has many industrial applications, including use as a precursor for polyamide polymers (e.g., nylon 6,10), dibasic polyesters, lubricants, and three-dimensional scaffold materials. Further applications, either by itself or as a precursor, include uses as a pH regulating agent, heat stabilizer, hydraulic fluid, and plasticizer (see refs. 1-3).

Sebacic acid is also a precursor to dibutyl sebacate, also known as dibutyl decanedioate, CAS Registry number 109-43-3, which can be used as a plasticizer, for food packaging, as a lubricant, as a synthetic flavoring additive, and can be used itself or as a precursor for use as a softener and conditioner (emollient), film former, fragrance, softener and conditioner (hair conditioning and skin conditioning), deodorizer, and solvent (see refs. 1-3).

Currently, the commercial synthesis of sebacic acid typically proceeds from ricinoleic acid, isolated from castor oil with the byproduct 2-octanol, with China contributing 90% of global trade (see ref. 1). Such production requires high temperature and alkaline conditions, carrying environmental risks (see refs. 1, 7). The large percentage of trade located in one country, along with the assumed difficulties of transferring that production method to other countries due to environmental concerns, indicates a supply risk for sebacic acid and therefore dibutyl sebacate as well.

A need exists for new techniques for the production of sebacic acid and dibutyl sebacate, preferably using environmentally-friendly techniques.

### SUMMARY OF THE INVENTION

Reaction of an oil (such as soybean oil) with one of a multiple of different lipases and Propionibacterium acnes linoleic acid isomerase (PAI), followed by reaction with an oxidant, can yield sebacic acid. Reaction of sebacic acid with 1-butanol and one of a multiple of different lipases can yield dibutyl sebacate. The lipases can either be on a solid scaffold or free in solution. Useful byproducts of these reactions could include caproic acid, butyl caproate, oxalic acid, and dibutyl oxalate.

In one embodiment, a method of making sebacic acid includes providing a starting material comprising linoleic acid; treating the starting material with a first lipase to obtain free linoleic acid; treating the free linoleic acid with a linoleic acid isomerase obtained from Propionibacterium acnes to obtain trans-10, cis-12 conjugated linoleic acid (CLA); and treating the CLA with an oxidant to obtain sebacic acid.

In another embodiment, a method of making sebacic acid includes providing a starting material comprising vegetable oil, linoleic acid, and/or trans-10, cis-12 conjugated linoleic acid; treating the starting material with a first lipase to obtain free linoleic acid; treating the free linoleic acid with a linoleic acid isomerase obtained from Propionibacterium acnes to obtain trans-10, cis-12 conjugated linoleic acid (CLA); and then treating the CLA with KMnO₄, sodium thiocyanate, and acid to obtain sebacic acid.

In a further embodiment, a method of making sebacic acid includes providing a starting material comprising vegetable oil, linoleic acid, and/or trans-10, cis-12 conjugated linoleic acid; treating the starting material with a first lipase to obtain free linoleic acid; treating the free linoleic acid with a linoleic acid isomerase obtained from Propionibacterium acnes to obtain trans-10, cis-12 conjugated linoleic acid (CLA); and treating the CLA with RuCl₃ and/or NaIO₄ to obtain sebacic acid.

In an added embodiment, a method of making sebacic acid includes providing a starting material comprising vegetable oil, linoleic acid, and/or trans-10, cis-12 conjugated linoleic acid; and treating the starting material with RuCl₃ and/or NaIO₄ to obtain sebacic acid.

In an alternative embodiment, a method of making sebacic acid includes providing a starting material comprising vegetable oil, linoleic acid, and/or trans-10, cis-12 conjugated linoleic acid; treating the starting material with a first lipase to obtain free linoleic acid; and treating the free linoleic acid with KMnO₄, sodium thiocyanate, and acid to obtain sebacic acid.

In yet another embodiment, a method of making sebacic acid includes providing a starting material comprising vegetable oil, linoleic acid, and/or trans-10, cis-12 conjugated linoleic acid; treating the starting material with a first lipase to obtain free linoleic acid; and treating the free linoleic acid RuCl₃ and/or NaIO₄ to obtain sebacic acid.

In still another embodiment, a method of making sebacic acid includes providing a starting material comprising vegetable oil, linoleic acid, and/or trans-10, cis-12 conjugated linoleic acid; treating the starting material with a linoleic acid isomerase obtained from Propionibacterium acnes to obtain trans-10, cis-12 conjugated linoleic acid (CLA); and treating the CLA with KMnO₄, sodium thiocyanate, and acid to obtain sebacic acid.

In a yet further embodiment, a method of making sebacic acid includes providing a starting material comprising vegetable oil, linoleic acid, and/or trans-10, cis-12 conjugated linoleic acid; treating the starting material with a linoleic acid isomerase obtained from Propionibacterium acnes to obtain trans-10, cis-12 conjugated linoleic acid (CLA); and treating the CLA with RuCl₃ and/or NaIO₄ to obtain sebacic acid.

In an added embodiment, a method of making sebacic acid includes providing a starting material comprising vegetable oil, linoleic acid, and/or trans-10, cis-12 conjugated linoleic acid; and treating the starting material with KMnO₄, sodium thiocyanate, and acid to obtain sebacic acid.

In yet another embodiment, sebacic acid (such as that obtained as described herein) is treated with a second lipase and 1-butanol to obtain dibutyl sebacate, wherein the second lipase is the same or different from the first lipase.

In another embodiment, a method of making sebacic acid includes providing a starting material comprising a fatty acid or a fatty acid-glycerin adduct, wherein the fatty acid has an alkene group isomerizable to the 10 position; treating the starting material with a first lipase to obtain the fatty acid in free form, when the starting material is the fatty acid-glycerin adduct; treating the fatty acid with a fatty acid isomerase that converts the alkene to the 10 position; and treating the fatty acid having the 10 position alkene with an oxidant to obtain sebacic acid.

### BRIEF DESCRIPTION OF DRAWINGS

A more complete appreciation will be readily obtained by reference to the following Description of the Example Embodiments and the accompanying drawings.

FIG. 1 depicts an exemplary overall reaction scheme. Vegetable oil, such as soybean oil, may contain triglycerides, diglycerides, and free fatty acids. To improve the reactivity of the isomerase PAI to the fatty acid chain, first a lipase is used to liberate the fatty acid chains from the triglyceride and diglycerides. In particular, the fatty acid of interest here is linoleic acid. Glycerol is a byproduct of this process. In the second step, PAI is used to isomerize the 9-position alkene to the 10-position on the fatty acid chain. This is important to ensure that one of the oxidation products is 10 carbons long (sebacic acid). In the third step, an oxidant is used to cleave the 10-position alkene resulting in either an aldehyde or a carboxylic acid; the aldehyde is eventually converted into a carboxylic acid (e.g., by addition of acid, by reaction with the oxidant, and/or by reaction with H₂O). Assuming full oxidation of the diene, byproducts would include oxalic acid and caproic acid. In the optional fourth step, lipase is used to esterify sebacic acid with 1-butanol to form dibutyl sebacate. If byproducts oxalic acid and caproic acid remain, these could be esterified as well. Note that other compounds could be formed by incomplete reaction. For example, linoleic acid that was not isomerized could be oxidized into azelaic acid.

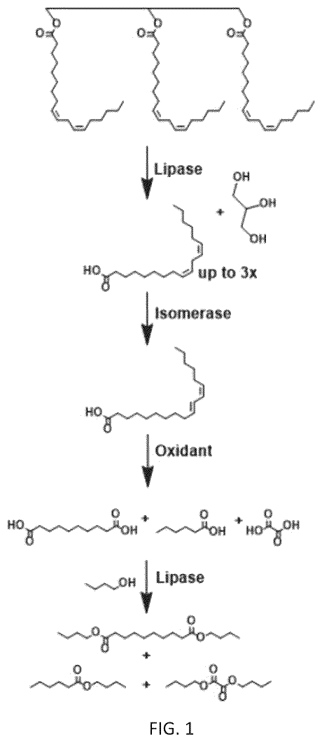

FIGS. 2A-2F provide LC/MS chromatograms indicating the chemoenzymatic pathway can produce sebacic acid from plant oil using lipase, PAI, KMnO₄, sodium thiocyanate, and acid. FIG. 2A is the sebacic acid standard. FIG. 2B is sebacic acid seen from these conditions (PAI, KMnO₄, and acid) without lipase. FIG. 2C is the reaction from lipase L1, which is a lipase immobilized on Immobead 150 from Rhizopus oryzae, ≥300 U/g. FIG. 2D is the reaction from lipase L2, which is recombinant lipase on acrylic resin, the lipase having been expressed in Aspergillus niger, ≥5,000 U/g. FIG. 2E is the reaction from lipase L3, which is from Rhizopus oryzae, powder, ≥30,000 U/g. FIG. 2F is the reaction from lipase L4 which is from Thermomyces lanuginosus. The reactions L1-L4 all had areas between ˜1.2×10⁶-1.5×10⁶, with peaks corresponding to the retention time and m/z of the sebacic acid standard. Conversely, the L0 control had much less area for sebacic acid, ˜7.1×10⁴. Therefore, sebacic acid was produced with reactions with lipase, PAI, KMnO₄, and acid, and sebacic acid was produced with all lipases indicated.

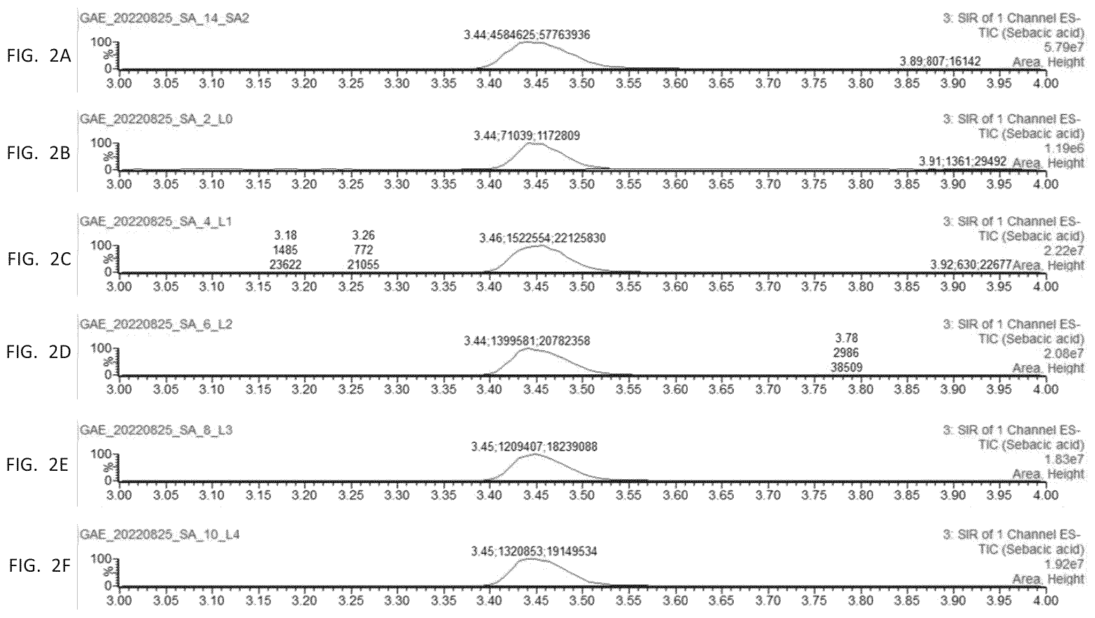

FIGS. 3A-3F provide LC/MS chromatograms indicating that very little, if any, sebacic acid is seen from plant oil treated with lipases but without PAI and without KMnO₄. FIG. 3A is the sebacic acid standard. FIG. 3B is sebacic acid seen from these conditions without lipase. FIGS. 3C-3F are sebacic acid seen from these conditions with the lipases L1-L4. The areas for reactions L1-L4, −PAI, −KMnO₄ had areas ˜2.1×10³-6.7×10³ and the control had area ˜2.3×10³, much lower than the reactions with all components in FIGS. 2C-2F.

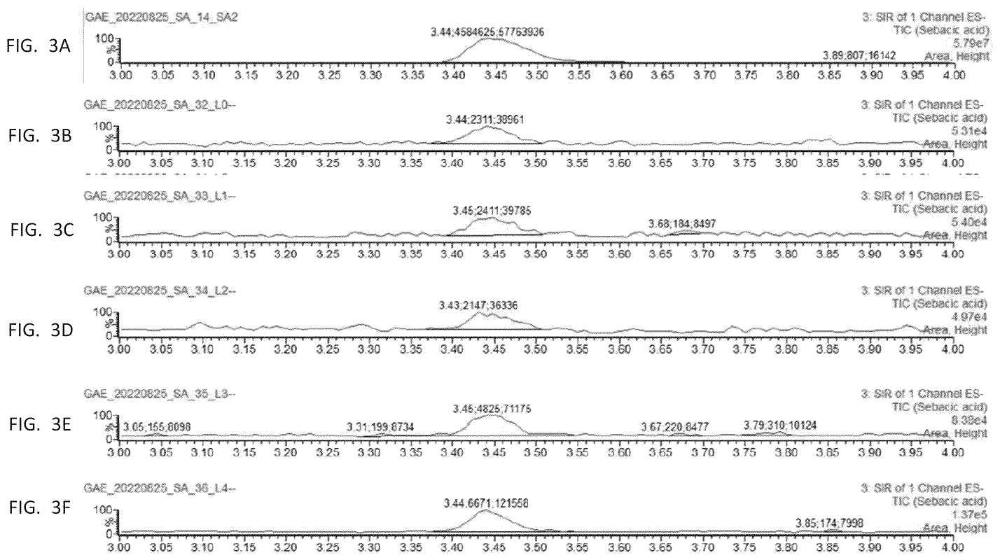

FIGS. 4A-4F provide LC/MS chromatograms indicating that very little sebacic acid is seen from plant oil treated with lipases and with PAI but without KMnO₄. FIG. 4A is the sebacic acid standard. FIG. 4B is sebacic acid seen from these conditions without lipase. FIGS. 4C-4F are sebacic acid seen from these conditions with different lipases L1-L4. The areas for reactions L1-L4-, +PAI, −KMnO₄ had areas ˜2.9×10³-1.7×10⁴ and the control had area ˜5.5×10³, much lower than the reactions with all components in FIGS. 2C-2F.

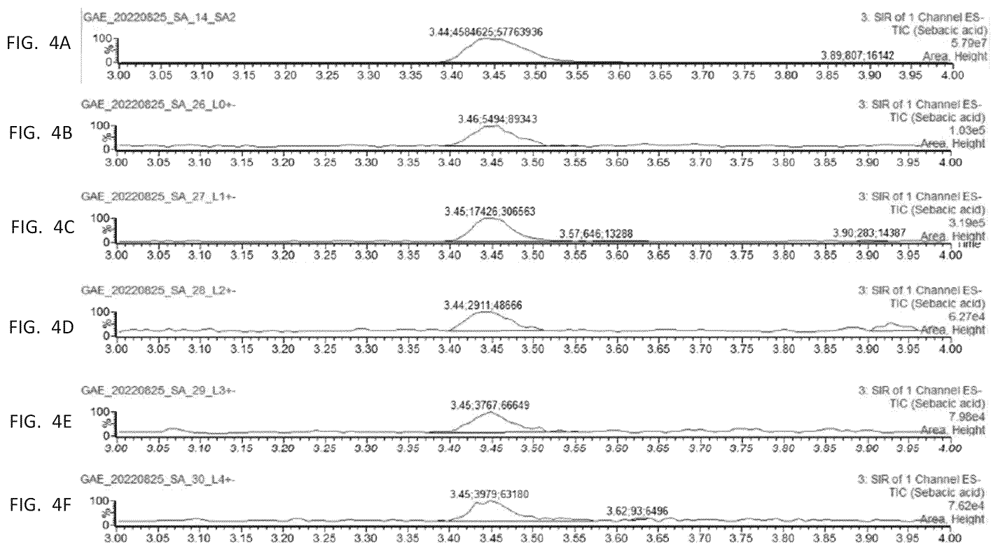

FIGS. 5A-5F provide LC/MS chromatograms indicating that some sebacic acid is seen from plant oil treated with lipases and with KMnO₄ but without PAI, although less than with PAI. FIG. 5A is the sebacic acid standard. FIG. 5B is sebacic acid seen from these conditions without lipase. FIGS. 5C-5F are sebacic acid seen from these conditions with different lipases L1-L4. The areas for reactions L1-L4, −PAI, +KMnO₄ had areas ˜1.3×10⁵-1.8×10⁵ and the control had area ˜9.4×10³, lower than the reactions with all components in FIGS. 2C-2F but higher than FIGS. 3C-3F and FIGS. 4C-4F.

FIGS. 6A-6F provide LC/MS chromatograms indicating that some sebacic acid is seen from t10,c12-CLA with RuCl₃ and NaIO₄. FIG. 6A is the sebacic acid standard. FIG. 6B is sebacic acid seen without RuCl₃ and without NaIO₄. FIG. 6C is sebacic acid seen with RuCl₃ but without NaIO₄. FIG. 6D is sebacic acid seen without RuCl₃ but with NaIO₄. FIG. 6E is sebacic acid seen with RuCl₃ and NaIO₄. FIG. 6F is sebacic acid seen with RuCl₃ and additional NaIO₄. The areas seen in FIG. 6E (˜1.6×10⁴) and FIG. 6F (˜7.3×10⁵) are greater than areas seen in FIGS. 6B-6D (˜1.1×10³-˜2.1×10³), indicating that reactions with both RuCl₃ and NaIO₄ are most productive. The areas seen in FIG. 6C (˜2.0×10³) and FIG. 6D (˜2.1×10³) are greater than the area in FIG. 6B (˜1.1×10³), indicating that either RuCl₃ or NaIO₄ is sufficient for additional reaction over the control.

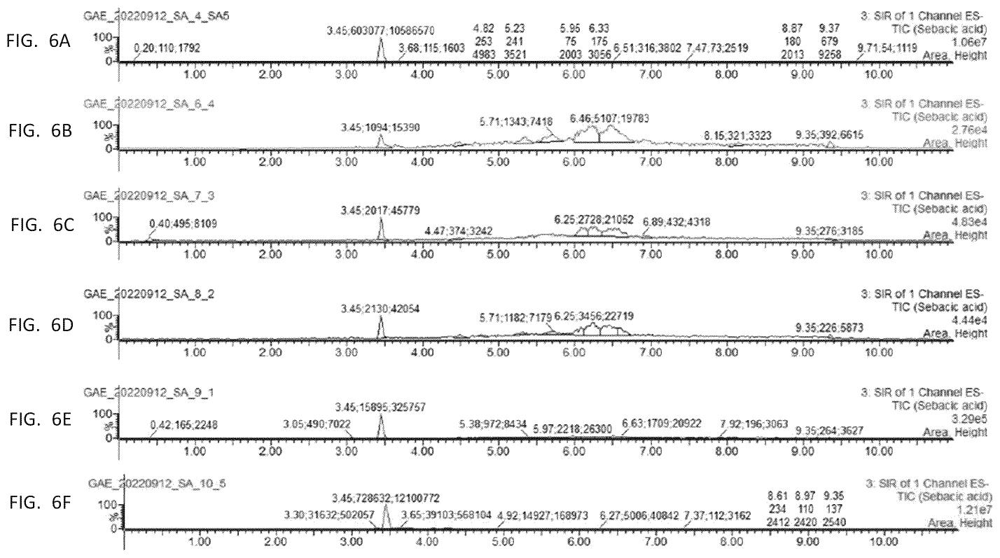

FIGS. 7A-7G provide LC/MS chromatograms indicating that some dibutyl sebacate is seen from sebacic acid with lipases and 1-butanol. FIG. 7A is the dibutyl sebacate standard. FIG. 7B is a control without lipase. FIGS. 7C-7G are the reactions with different lipases (L1-L5). The areas seen in FIGS. 7C-7G (˜5.4×10⁶-˜2.3×10⁷) are higher than the area for the control reaction in FIG. 7B (˜6.7×10⁴), indicating that these reactions produced dibutyl sebacate

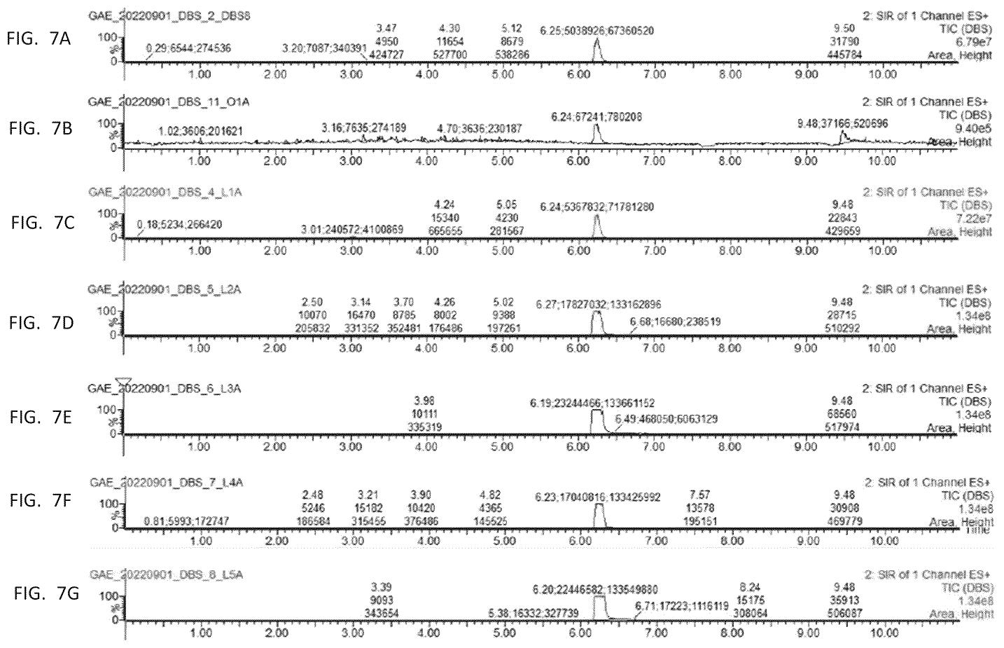

FIGS. 8A-8G provide LC/MS chromatograms indicating that some dibutyl sebacate is seen from sebacic acid with lipases and 1-butanol, in the presence of hexanes. FIG. 8A is the dibutyl sebacate standard. FIG. 8B is a control without lipase. FIGS. 8C-8G are the reactions with different lipases. The areas seen in FIGS. 8C-8G (˜2.3×10⁷-˜3.1×10⁷) are higher than the area for the control reaction in FIG. 8B (˜9.8×10⁴), indicating that these reactions produced dibutyl sebacate.

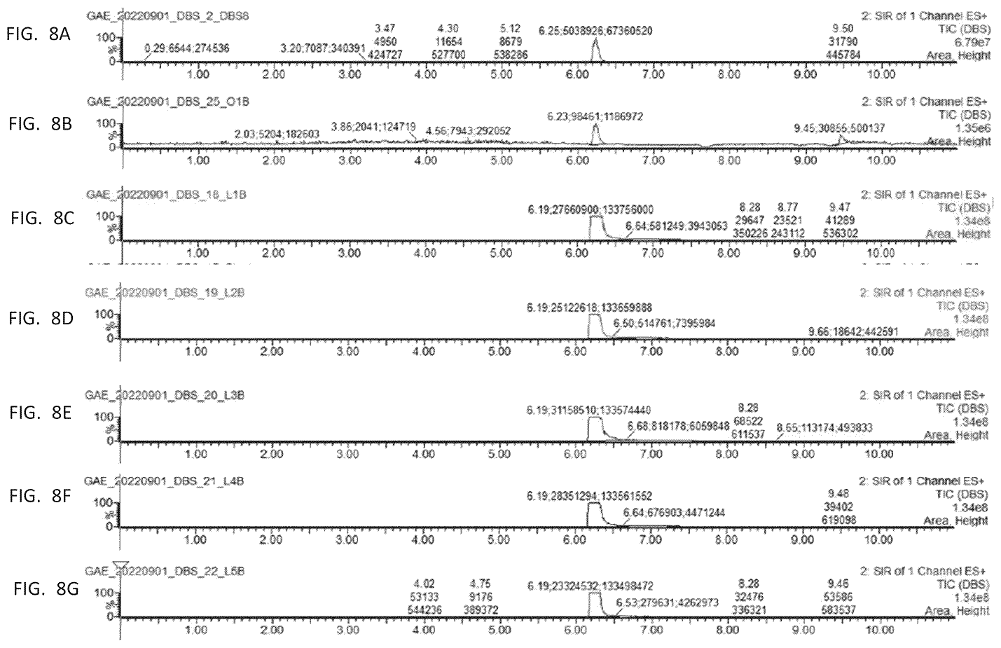

FIGS. 9A-9F provide selected negative mode spectra from LC/MS analysis of sebacic acid production, corresponding to the chromatograms in FIG. 2. Sebacic acid expected m/z ratio for [M−H]−=201.11. Spectra zoomed in to see m/z peak and mass envelope. FIG. 9A corresponds to FIG. 2A. FIG. 9B corresponds to FIG. 2B. FIG. 9C corresponds to FIG. 2C. FIG. 9D corresponds to FIG. 2D. FIG. 9E corresponds to FIG. 2E. FIG. 9F corresponds to FIG. 2F.

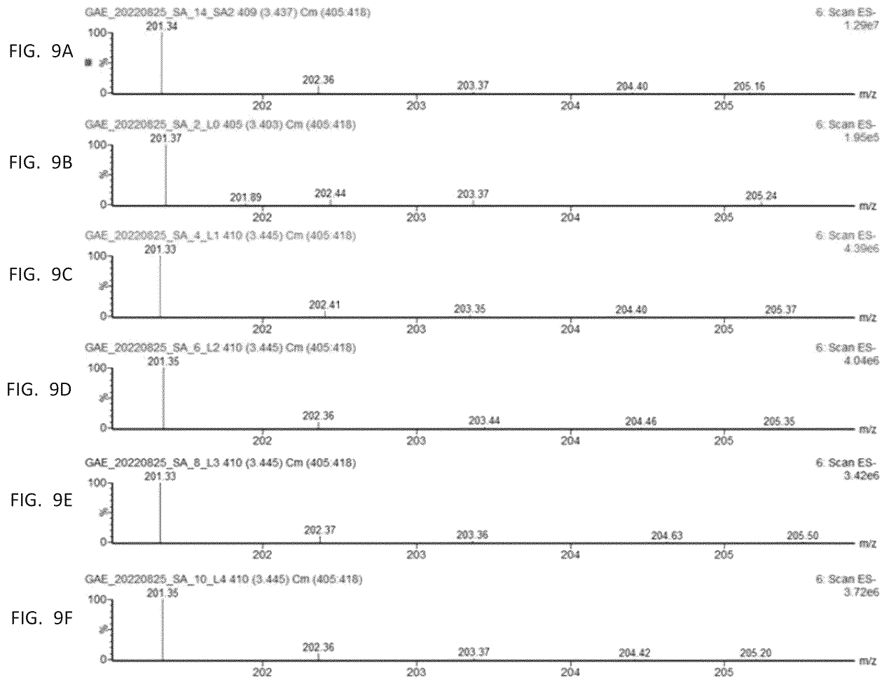

FIGS. 10A-10F provide selected negative mode spectra from LC/MS analysis of sebacic acid production, corresponding to the chromatograms in FIG. 6. Sebacic acid expected m/z ratio for [M−H]−=201.11. Spectra zoomed in to see m/z peak and mass envelope. FIG. 10A corresponds to FIG. 6A. FIG. 10B corresponds to FIG. 6B. Note that no peak is seen near 201.11 m/z. This corresponds with low detection as shown in FIG. 6B, which was the control without RuCl₃ and without NaIO₄. FIG. 10C corresponds to FIG. 6C. FIG. 10D corresponds to FIG. 6D. FIG. 10E corresponds to FIG. 6E. FIG. 10F corresponds to FIG. 6F.

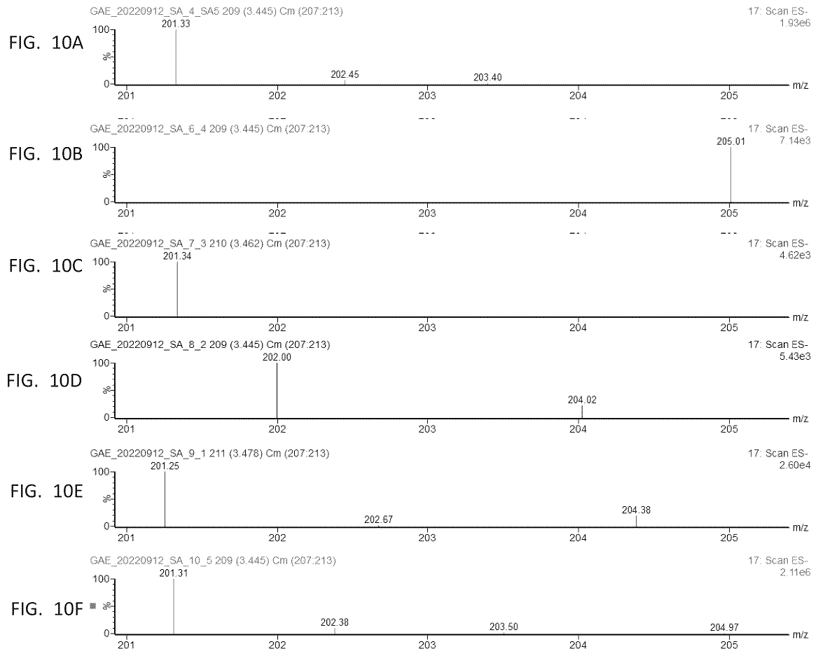

FIGS. 11A-11G provide selected positive mode spectra from LC/MS analysis of dibutyl sebacate production, corresponding to the chromatograms in FIG. 7. Dibutyl sebacate expected m/z ratio for [M+H]+=315.25. Spectra zoomed in to see m/z peak and mass envelope. FIG. 11A corresponds to FIG. 7A. FIG. 11B corresponds to FIG. 7B. FIG. 11C corresponds to FIG. 7C. FIG. 11D corresponds to FIG. 7D. FIG. 11E corresponds to FIG. 7E. FIG. 11F corresponds to FIG. 7F. FIG. 11G corresponds to FIG. 7G.

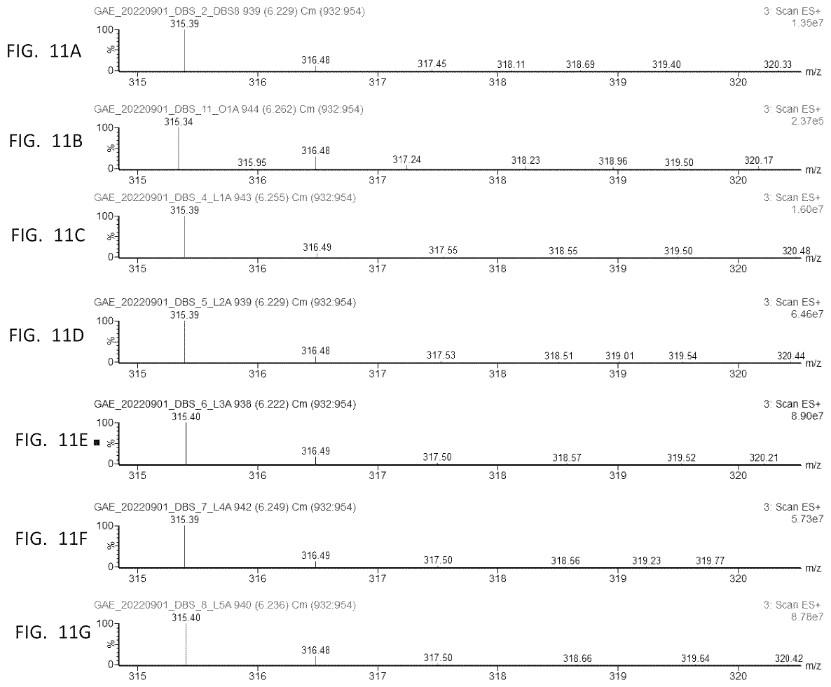

FIGS. 12A-12F provide selected positive mode spectra from LC/MS analysis of dibutyl sebacate production, corresponding to the chromatograms in FIG. 8. Dibutyl sebacate expected m/z ratio for [M+H]+=315.25. Spectra zoomed in to see m/z peak and mass envelope. Note that FIG. 11A corresponds to FIG. 8A (as well as 7A). FIG. 11A corresponds to FIG. 8B. FIG. 11B corresponds to FIG. 8C. FIG. 11C corresponds to FIG. 8D. FIG. 11D corresponds to FIG. 8E. FIG. 11E corresponds to FIG. 8F. FIG. 11F corresponds to FIG. 8G.

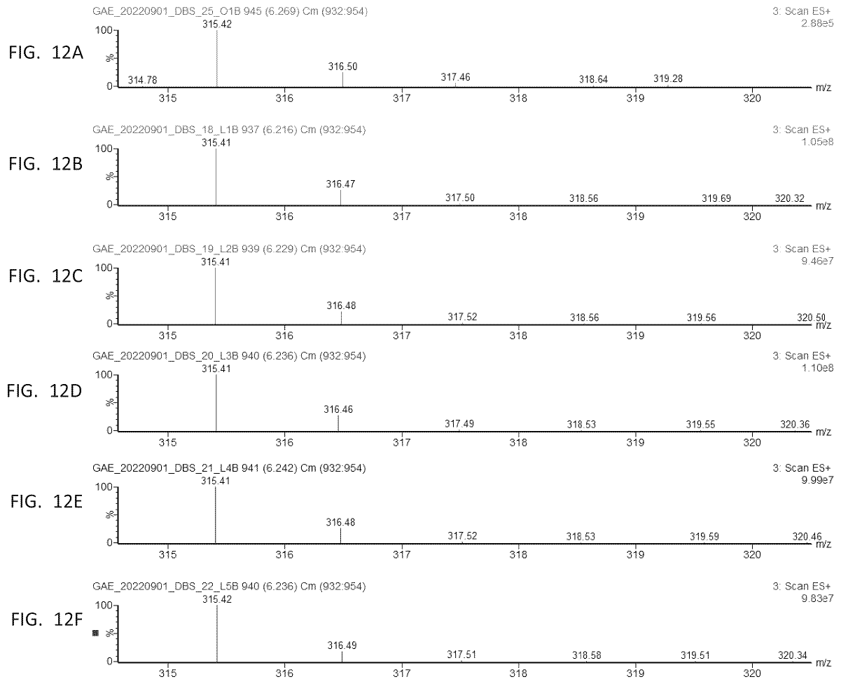

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of +10% of that stated.

As used herein, the term “starting material comprising linoleic acid” includes triglycerides and diglycerides from which linoleic acid can be liberated via a lipase, as well as linoleic acid as a free fatty acid. Examples of such starting material include vegetable oils such as soy, corn, and/or canola oil.

### Overview

As described herein, reaction of vegetable oil (for example, soybean oil) with one of a multiple of different lipases and linoleic acid isomerase from Propionibacterium acnes (PAI), followed by reaction with an oxidant, can yield sebacic acid. Reaction of sebacic acid with 1-butanol and one of a multiple of different lipases can yield dibutyl sebacate. The lipases can either be on a solid scaffold or free in solution. Byproducts of these reactions could include caproic acid, butyl caproate, oxalic acid, and dibutyl oxalate. Further compounds of use could be produced from incomplete reactions within the pathway.

Previous work (see ref. 8) has indicated that Rhizopus oryzae lipase can convert plant oil triglycerides to linoleic acid (LA, Cas Registry number 60-33-3) and that Propionibacterium acnes linoleic acid isomerase (PAI) can convert LA to trans-10, cis-12 conjugated linoleic acid (t10,c12-CLA, Cas Registry number 2420-56-6). Note that the same PAI protein sequence has also been indicated as coming from Cutibacterium, since Cutibacterium were formerly known as Propionibacterium.

Furthermore, oxidants can yield cleavage of a diene, resulting in two carboxylic acids. Examples of these oxidants include potassium permanganate (KMnO4, Cas Registry Number 7722-64-7) followed by acidification, RuCl3 with NaIO4, and ozone (see refs. 9-11). Moreover, lipases can esterify carboxylic acids, combining a carboxylic acid and an alcohol (see refs. 12, 13).

### EXAMPLES

Vegetable oil, which can contain linoleic acid triglycerides, and which may contain free linoleic acid, was subjected to one of a variety of lipases. The lipases were either immobilized onto a scaffold or free in solution, with the expectation they would hydrolyze the triglyceride to release free linoleic acid. To this solution was added PAI (SEQ ID NO: 1), an isomerase that is known to isomerize linoleic acid primarily to trans-10, cis-12 conjugated linoleic acid (t10,c12-CLA, or CLA). It is important that the isomerase catalyzes this reaction, rather than the other possibility of isomerizing to the 9,11 conjugated linoleic acid, to ensure the alkene is in the correct location for cleavage and oxidation to sebacic acid. After reaction with PAI, an oxidant was added and allowed to react. In one case, KMnO4 was added and allowed to react, followed by addition of sodium thiocyanate to quench any unreacted KMnO4, acidification, and extraction of sebacic acid. In another case, RuCl3 (ruthenium chloride) and/or NaIO4 (sodium periodate) were added and allowed to react, followed by extraction of sebacic acid. These chemistries were used to cleave the alkene(s) and oxidize the intermediate to result in sebacic acid. Production of sebacic acid was confirmed by LC/MS analysis. Lipases were used to convert sebacic acid to dibutyl sebacate, using 1-butanol as the other co-substrate, with or without hexanes present. Multiple lipases were able to release free linoleic acid, and multiple lipases were able to esterify sebacic acid to dibutyl sebacate, portending the potential use of many different lipases for these steps. Multiple oxidants were able to cleave and oxidize t10,c12-CLA, portending the potential use of many different oxidants for this step.

To elaborate, pH buffer and bovine albumin were added to vegetable oil to help solubilize it. This was applied to four reactions with different lipases and an additional control with no lipase (L0). Reactions used the following lipases: (L1)=Lipase, immobilized on Immobead 150 from Rhizopus oryzae, ≥300 U/g (Sigma Aldrich); (L2)=Lipase acrylic resin, recombinant, expressed in Aspergillus niger, ≥5,000 U/g (Sigma Aldrich); (L3)=Lipase from Rhizopus oryzae, powder, ≥30,000 U/g (Sigma Aldrich); and (L4)=Lipase from Thermomyces lanuginosus (Sigma Aldrich). These reactions were allowed to proceed at 30° C., at 130 rpm, in an incubator, but other conditions would likely also result in reaction, preferably not to exceed 100° C.

The above reactions and controls (L0-L4, +PAI and L0-L4, −PAI) were each split in two. One set received KMnO4 (L0-L4, +PAI, +KMnO4; L0-L4, −PAI, +KMnO4) and one set did not receive KMnO4 and acted as a control (L0-L4, +PAI, −KMnO4; L0-L4, −PAI, −KMnO4). The reactions and controls were allowed to proceed at 30° C., at 130 rpm, in an incubator, but other conditions would likely also result in reaction.

To all reactions and controls that had received KMnO4 was added sodium thiocyanate to reduce any unreacted KMnO4. All reactions and controls that had received KMNO4 received HCl to acidify the solutions. Ethyl acetate was added to extract sebacic acid from those solutions that contained it. A portion of the ethyl acetate partition was removed and dried under vacuum. Methanol and water were added to the resulting material, and subjected to LC/MS analysis. Analysis was performed to indicate those samples containing sebacic acid and a relative amount between samples.

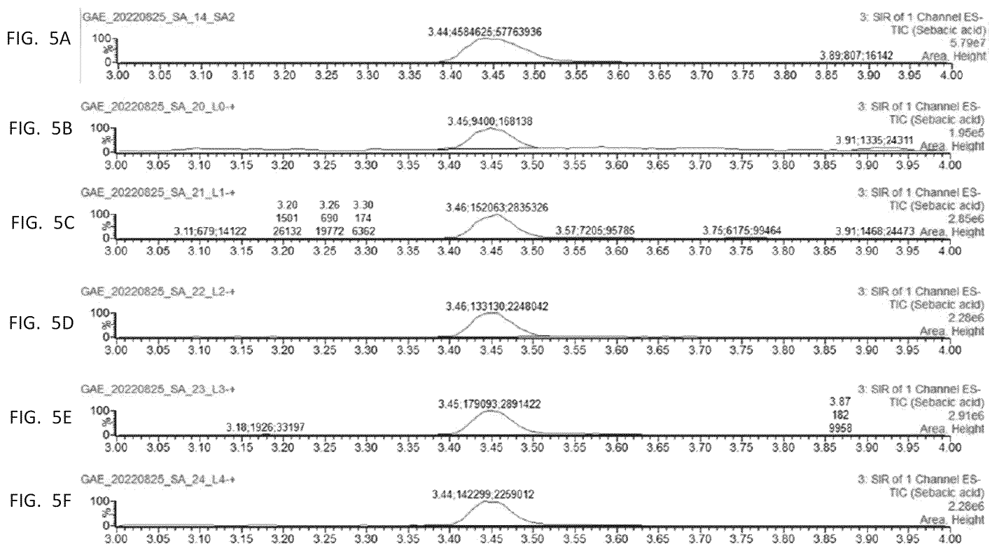

Given the results of FIGS. 5A-5F, the chemical reaction (i.e., KMnO4, sodium thiocyanate, and acid) appears sufficient to produce sebacic acid; neither PAI nor lipase is required. A small amount of t10,c12-CLA already present in the vegetable oil, or a small amount of t10,c12-CLA produced under these chemical conditions, cannot be ruled out as a possible reason for production of sebacic acid without lipase or PAI. However, the combination of lipase, PAI, KMnO4, sodium thiocyanate, and acid produces a much higher amount of sebacic acid. Therefore, one embodiment is the use of KMnO4, sodium thiocyanate, and acid to produce sebacic acid from vegetable oil, linoleic acid, and/or conjugated linoleic acid. A second embodiment is the use of KMnO4, sodium thiocyanate, acid, and lipase to produce sebacic acid from vegetable oil, linoleic acid, and/or conjugated linoleic acid. A third embodiment is the use of KMnO4, sodium thiocyanate, acid, and PAI to produce sebacic acid from vegetable oil, linoleic acid, and/or conjugated linoleic acid. A fourth embodiment is a combination of lipase, PAI, KMnO4, sodium thiocyanate, and acid to produce sebacic acid from vegetable oil, linoleic acid, and/or conjugated linoleic acid.

To the four types of lipases referenced above, as well as (L5)=Lipase (MP Biomedical), was added buffer, sebacic acid dissolved in dimethyl sulfoxide (DMSO), and 1-butanol. Control reactions did not contain lipase. A similar set of reactions were conducted with hexanes added. Reactions and controls were allowed to proceed at ambient temperature, on an orbital shaker, but other conditions would likely also result in reaction, preferably not to exceed 100° C.

Reactions were conducted of t10,c12-CLA, RuCl3, and NaIO₄ in an aqueous/organic mixture. Controls were conducted without RuCl3, or without NaIO4, or without RuCl3 and without NaIO4. A further reaction was conducted with additional NaIO4. Reactions and controls were allowed to proceed at ambient temperature, on an orbital shaker, but other conditions would likely also result in reaction.

### FURTHER EMBODIMENTS

Further embodiments could be conceived of as the following: (1) other lipases, such as those from different source organisms or expressed by different organisms, and/or variants of lipases, can be used for this technique; (2) other scaffolds for enzymes, such as Nickel-nitrilotriacetic acid (Ni-NTA) resins, agarose beads, nanoparticles, and silica particles, can be used to scaffold enzymes for this technique; (3) other isomerases, such as those from different source organisms or expressed by different organisms, and/or variants of PAI, can be used for this technique, if the isomerase results in the correct position of the alkenes (positions 10, 12); (4) other oxidants, such as ozone, which can cleave alkenes and result in carboxylic acids, can be used for this technique; and (5) other substrates that contain an alkene in the correct position (i.e., position 10), or that can be isomerized such that the resultant product has an alkene in the correct position (e.g., oleic acid or linolenic acid), whether as free acids or as parts of larger compounds (e.g., triglycerides), can be used for this technique.

In addition to sebacic acid and dibutyl sebacate, the following compounds that could be byproducts of a method to produce sebacic acid and dibutyl sebacate, can have multiple uses: caproic acid, capronic acid, 1-hexanoic acid, CAS Registry number 142-62-1; butyl caproate, butyl ester hexanoic acid, CAS Registry number 626-82-4; oxalic acid, ethanedioic acid, CAS Registry number 144-62-7; dibutyl oxalate, 1,2-dibutyl ester ethanedioic acid, CAS Registry number 2050-60-4. Caproic acid itself or as a precursor can be used as a feed additive, antimicrobial, promoter of plant growth/plant additive, pH regulating agent, cleaning agent, emulsifier, fragrance, deodorizer, solvent, surfactant, lubricant, pharmaceuticals, and for flavoring. Butyl caproate itself or as a precursor can be used for flavoring and fragrance. Oxalic acid itself or as a precursor can be used for cleaning, as a stripper, a pH regulating agent (acid), a chelating agent, and a fragrance. Dibutyl oxalate can be used itself or as a precursor as a chelating agent, softener and conditioner (hair conditioning), plasticizer, and solvent (see refs. 2, 3, 14).

The linoleic acid isomerase obtained from Propionibacterium acnes, termed PAI, can be a native protein or genetically modified. The genetic modifications can include codon optimization and/or the addition of a histidine or other tag for purification.

### Advantages

The method of using enzymes and/or chemistry (i.e., chemoenzymatic) as described herein to produce sebacic acid and dibutyl sebacate results in many advantages.

This technique can use vegetable oils containing linoleic acid and/or linoleic acid derivatives (e.g., linoleic acid triglyceride) to produce sebacic acid and dibutyl sebacate, providing an alternative feedstock to ricinoleic acid and castor oil. This mitigates supply risks and/or offers a feedstock that may be more economically advantageous. Additional starting materials comprising linoleic acid can include agricultural oils derived from articles including, without limitation, nuts, seeds, egg yolk, lard, cocoa butter, butter, and various oils: sunflower, safflower, canola, grape seed, poppyseed, hemp, wheat germ, cottonseed, walnut, sesame, rice bran, pistachio, peanut, linseed, olive, palm, macadamia, and coconut (see refs. 15-22).

Moreover, this method can be a greener, more environmentally-friendly method of producing sebacic acid and dibutyl sebacate, over other routes. This is due primarily to the use of enzymes, which can work in water-based solvents (aqueous) near ambient temperature and pressure, and which themselves can be produced in a green, environmentally-friendly manner (e.g., cell culture). Yet, as demonstrated above, the technique tolerates the presence of hexanes as representative non-aqueous solvent contaminants.

The use of scaffolding for enzymes can facilitate enzyme stability, activity, recovery, reuse, and purification of sebacic acid and dibutyl sebacate. For example, the immobilized lipase could be easily separated from the reaction after releasing linoleic acid from glycerol (e.g., by centrifugation) then used in the last step of esterification. In another example, all immobilized enzymes could be separated from the reaction for continual reuse.

The technique could be amenable to being conducted in a series of flow connected chambers, especially with the use of immobilized enzymes, which could have distinct advantages in production such as controllability, temperature regulation, and minimization of waste.

The inclusion of a His-tag on the PAI enzyme would make this enzyme amenable to immobilization (e.g., onto Nickle-nitrilotriacetic acid functionalized resins or onto quantum dots with a ZnS shell). Note that the PAI enzyme could also be immobilized by other means (e.g., reaction with Immobead 150 resin).

Byproducts from these reactions are glycerol, CAS Registry number 56-81-5; caproic acid, capronic acid, 1-hexanoic acid, CAS Registry number 142-62-1; butyl caproate, butyl ester hexanoic acid, CAS Registry number 626-82-4; oxalic acid, ethanedioic acid, CAS Registry number 144-62-7; dibutyl oxalate, 1,2-dibutyl ester ethanedioic acid, CAS Registry number 2050-60-4. These products could be beneficial as end products. Additionally, other side products may result from some incomplete reactions steps, which could be beneficial end products (e.g., azelaic acid, nonanedioic acid, CAS Registry number 123-99-9, which could result from incomplete isomerization of linoleic acid followed by oxidation).

The technique described herein is expected to be amenable to the production of analogs of sebacic acid and dibutyl sebacate, as long as the enzymes used tolerate the initial substituted substrate (i.e., substrate analog) and the chemistry involved does not destroy the molecule. For example, other esters of sebacic acid could be produced with alternative alcohols (e.g., ethanol), or a fluorine-substituted linoleic acid may produce fluorine-substituted sebacic acid or dibutyl sebacate.

This method is flexible regarding the esterification. The oxidation of the alkene could be stopped at the point of forming an aldehyde instead of a carboxylic acid. This could be followed by reaction with an alcohol to form the ester, instead of the use of lipase (e.g., using KOH and I2, or using Br2 and H2O2 (30% aqueous)). In another example, the carboxylic acid formed from oxidation of the alkene could be reacted with an alcohol to form the ester (e.g., using sulfuric acid and heat) (see refs. 23-26).

### Concluding Remarks

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

### REFERENCES

1. Jeon, W-Y., et al. Green Chem. 21, 6491, 2019. DOI: 10.1039/c9gc02274k
2. Dionisio, K. L., et al. Scientific Data 5, 180125, 2018. DOI: 10.1038/sdata.2018.125
3. https://comptox.epa.gov/dashboard/, accessed Sep. 14, 2022
4. “Toxicological Profile for Otto Fuel II and Its Components” U.S. Department of Health and Human Services, Public Health Service, Agency for Toxic Substances and Disease Registry, June 1995.
5. U.S. Pat. No. 4,026,739 “Liquid monopropellants of reduced shock sensitivity and explodability,” issued May 31, 1977
6. Powell, S., et al. Anaerobe. 4, 95-102, 1998. DOI: 10.1006/anae.1997.0141
7. Takeuchi, M., et al. Bioscience, Biotechnology, and Biochemistry. 80, 2132-2137, 2016. DOI: 10.1080/09168451.2016.1200457
8. Zhang, Y., et al. Journal of Agricultural and Food Chemistry 65, 5322-5239, 2017. DOI: 10.1021/acs.jafc.7b00846.
9. Zimmermann, F., et al. Tetrahedron Letters 46, 3201-3203, 2005. DOI: 10.1016/j.tetlet.2005.03.052.
10. Erba, E. and La Rosa, C. Tetrahedron 72, 7975-7981, 2016. DOI: 10.1016/j.tet.2016.10.039
11. Kerenkan, A. E., et al. Catalysis Science & Technology 6, 971-987, 2016. DOI: 10.1039/c5cy01118c.
12. Swarts, J. W., et al. Biotechnology & Bioengineering 99, 855-861, 2008. DOI: 10.1002/bit.21650.
13. Hommes, A., et al. Industrial & Engineering Chemistry Research 58, 15432-15444, 2019. DOI: 10.1021/acs.iecr.9b02693.
14. Chen, W-S., et al. Environmental Science & Technology 51, 7159-7168, 2017. DOI: 10.1021/acs.est.6b06220.
15. Food and Agriculture Organization of the United Nations, https://www.fao.org/faostat/en/#data/QCL/visualize, accessed Mar. 23, 2022.
16. https://en.wikipedia.org/wiki/Corn_oil #cite_note-13
17. https://fdc.nal.usda.gov/fdc-app.html #/food-details/171029/nutrients, accessed Mar. 31, 2022
18. https://fdc.nal.usda.gov/fdc-app.html #/food-details/171411/nutrients, accessed Mar. 31, 2022)
19. Huth, P. J. et al. Adv. Nutr. 6, 674-693, 2015. DOI: 10.3945/an.115.008979
20. https://www.hsph.harvard.edu/nutritionsource/2014/11/05/dietary-linoleic-acid-and-risk-of-coronary-heart-disease/, accessed Sep. 21, 2022
21. https://www.news-medical.net/health/Oils-Rich-in-Linoleic-Acid.aspx, accessed Sep. 21, 2022
22. https://mtcapra.com/knowledge-base/linoleic-acid-content-in-various-oils/, accessed Sep. 21, 2022
23. G. A. Molander and D. J. Cooper. J. Org. Chem. 72, 3558-3560, 2007. DOI: 10.1021/jo070130r
24. Yamada, S., et al. Tetrahedron Letters. 33, 4329-4332, 1992.
25. Amati, A., et al. Organic Process Research & Development, 2, 261-269, 1998.
26. Wyrebek, P. et al. ACS Omega, 3, 18481-18488, 2018. DOI: 10.1021/acsomega.8b03119

What is claimed is:

1. A method of making sebacic acid, the method comprising:
providing a starting material comprising linoleic acid;
treating the starting material with a first lipase to obtain free linoleic acid;
treating the free linoleic acid with a linoleic acid isomerase obtained from Propionibacterium acnes to obtain trans-10, cis-12 conjugated linoleic acid; and
treating the trans-10, cis-12 conjugated linoleic acid with an oxidant to obtain sebacic acid.

2. The method of claim 1, further comprising treating the sebacic acid with a second lipase and 1-butanol to obtain dibutyl sebacate, wherein the second lipase is the same or different from the first lipase.

3. The method of claim 1, wherein said treating trans-10, cis-12 conjugated linoleic acid with an oxidant comprises the use of (1) a combination of KMnO₄ and acidification; (2) RuCl₃ and/or NaIO₄; or (3) ozone.

4. The method of claim 1, wherein a majority of the starting material is one or more vegetable oils.

5. The method of claim 1, wherein the linoleic acid isomerase has SEQ ID NO: 1.

6. A method of making sebacic acid, the method comprising:
providing a starting material comprising vegetable oil, linoleic acid, and/or trans-10, cis-12 conjugated linoleic acid; and
treating the starting material with KMnO4, sodium thiocyanate, and acid to obtain sebacic acid.

7. The method of claim 6, further comprising pretreating the starting material with a first lipase prior to said treating.

8. The method of claim 7, further comprising treating the sebacic acid with a second lipase and 1-butanol to obtain dibutyl sebacate, wherein the second lipase is the same or different from the first lipase.

9. A method of making sebacic acid, the method comprising:
providing a starting material comprising linoleic acid;
treating the starting material with a linoleic acid isomerase obtained from Propionibacterium acnes to obtain trans-10, cis-12 conjugated linoleic acid; and
treating the trans-10, cis-12 conjugated linoleic acid with an oxidant to obtain sebacic acid.

10. The method of claim 9, further comprising pretreating the starting material with a first lipase prior to said treating.

11. The method of claim 10, further comprising treating the sebacic acid with a second lipase and 1-butanol to obtain dibutyl sebacate, wherein the second lipase is the same or different from the first lipase.

12. The method of claim 9, wherein the linoleic acid isomerase has SEQ ID NO: 1.

13. A method of making sebacic acid, the method comprising:
providing a starting material comprising a fatty acid or a fatty acid-glycerin adduct:
wherein the fatty acid has an alkene group isomerizable to the 10 position;
treating the starting material with a first lipase to obtain the fatty acid in free form, when the starting material is the fatty acid-glycerin adduct;
treating the fatty acid with a fatty acid isomerase that converts the alkene to the 10 position; and
treating the fatty acid having the 10 position alkene with an oxidant to obtain sebacic acid.

14. The method of claim 13, further comprising treating the sebacic acid with a second lipase and 1-butanol to obtain dibutyl sebacate, wherein the second lipase is the same or different from the first lipase.

15. The method of claim 13, wherein said treating the fatty acid having the 10 position alkene with an oxidant comprises the use of (1) a combination of KMnO₄ and acidification; (2) RuCl₃ and/or NaIO₄; or (3) ozone.

16. The method of claim 1, wherein a majority of the starting material is one or more vegetable oils.
